SEQUENCE LISTING (28 sequences)

SEQ ID NO: 1 - DNA, 22 bp, Artificial
  sense primer of Bal1
gcagtgccactgactaccaaga

SEQ ID NO: 2 - DNA, 20 bp, Artificial
  Antisense primer of Bal1
tcctggacattgcattgcat

SEQ ID NO: 3 - DNA, 24 bp, Artificial
  sense primer of Npas2
gtatgcacagagccaagtgatgtt

SEQ ID NO: 4 - DNA, 22 bp, Artificial
  Antisense primer of Npas2
tgctcactgtgcagagatgttg

SEQ ID NO: 5 - DNA, 20 bp, Artificial
  sense primer of Rev-erb alpha
cgttcgcatcaatcgcaacc

SEQ ID NO: 6 - DNA, 18 bp, Artificial
  Antisense primer of Rev-erc alpha
gatgtggagtaggtgagg

SEQ ID NO: 7 - DNA, 25 bp, Artificial
  sense primer of Dbp
aatgacctttgaacctgatcccgct

SEQ ID NO: 8 - DNA, 25 bp, Artificial
  Antisense primer of Dbp
gctccagtacttctcatccttctgt

SEQ ID NO: 9 - DNA, 20 bp, Artificial
  sense primer of Rev-erb beta
acggattcccaggaacatgg

SEQ ID NO: 10 - DNA, 20 bp, Artificial
  Antisense primer of Rev-erb beta
cctccagtgttgcacaggta

SEQ ID NO: 11 - DNA, 22 bp, Artificial
  sense primer of Per3
ctgctccaactcagcttccttt

SEQ ID NO: 12 - DNA, 23 bp, Artificial
  Antisense primer of Per3
ttagacagcaaggctctggttct

SEQ ID NO: 13 - DNA, 25 bp, Artificial
  sense primer of Per1
caggctaaccaggaatattaccagc

SEQ ID NO: 14 - DNA, 25 bp, Artificial
  Antisense primer of Per1
cacagccacagagaaggtgtcctgg

SEQ ID NO: 15 - DNA, 20 bp, Artificial
  sense primer of Per2
ggcttcaccatgcctgttgt

SEQ ID NO: 16 - DNA, 23 bp, Artificial
  Antisense primer of Per2
ggagttatttcggaggcaagtgt

SEQ ID NO: 17 - DNA, 25 bp, Artificial
  sense primer of Cry1
cccaggcttttcaaggaatggaaca

SEQ ID NO: 18 - DNA, 25 bp, Artificial
  Antisense primer of Cry1
tctcatcatggtcatcagacagagg

SEQ ID NO: 19 - DNA, 23 bp, Artificial
  sense primer of Cry2
gggactctgtctattggcatctg

SEQ ID NO: 20 - DNA, 20 bp, Artificial
  Antisense primer of Cry2
gtcactctagcccgcttggt

SEQ ID NO: 21 - DNA, 22 bp, Artificial
  sense primer of Clock
cctatcctaccttggccacaca

SEQ ID NO: 22 - DNA, 20 bp, Artificial
  Antisense primer of Clock
tcccgtggagcaacctagat

SEQ ID NO: 23 - DNA, 20 bp, Artificial
  sense primer of Ck1 delta
ccagcctggaagacctgttc

SEQ ID NO: 24 - DNA, 18 bp, Artificial
  Antisense primer of Ck1 delta
tggccagcccaaagtcaa

SEQ ID NO: 25 - DNA, 21 bp, Artificial
  sense primer of Ck1 epsilon
ggatgtgaagcccgacaactt

SEQ ID NO: 26 - DNA, 20 bp, Artificial
  Antisense primer of Ck1 epsilon
tctcgacggctttgctcaat

SEQ ID NO: 27 - DNA, 18 bp, Artificial
  sense primer of Tim
acatgtgggcaatggctt

SEQ ID NO: 28 - DNA, 21 bp, Artificial
  antisense primer of tim
ctgctccacaaagtgaaaggt

(19) United States
(12) Patent Application Publication
(10) Pub. No.: US 2006/0073495 A1
(43) Pub. Date: Apr. 6, 2006

(54) Method for inspecting quality of sensor chip, sample evaluating method, DNA chip, and protein chip

(76) Inventors: Takuro Yamamoto, Kanagawa (JP); Toru Takumi, Kyoto (JP)

Correspondence Address: FROMMER LAWRENCE & HAUG LLP, 745 FIFTH AVENUE, NEW YORK, NY, 10151, US

(21) Appl. No.: 11/174,272

(22) Filed: Jul. 1, 2005

(30) Foreign Application Priority Data
Jul. 5, 2004 (JP) 2004-198619

(51) Int. Cl. C12Q 1/68; C12M 1/34

(52) U.S. Cl. 435/6; 435/287.2

(57) ABSTRACT

A method for inspecting a quality of a sensor chip using either of (1) and (2) indicated below as a judgment index is provided: (1) interrelations of a plurality of circadian rhythm control genes in respect of expression level thereof; and (2) time-lapse patterns of specified circadian rhythm control genes in respect of expression level thereof. A method of evaluating whether sampling and preparation of a sample to be subjected to detection of interaction between substances is proper is also provided wherein (1) or (2) defined above is used as a judgment index. DNA and protein chips useful for the methods are also provided.

### BACKGROUND OF THE INVENTION

This invention relates to a method for inspecting quality of a sensor chip, a sample evaluating method, a DNA chip and a protein chip. More particularly, the invention relates to a method for inspecting quality of a sensor chip using expression levels of circadian rhythm control genes as an index for judgment, and to a sample evaluating method provided for detecting interaction between substances. The invention also relates to a DNA chip and a protein chip, each using a specified substrate region as a region of a chip for quality inspection or a region for sample evaluation.

In the fields of drug discovery, clinical diagnosis, pharmacogenomics, forensic pathology, and the like, sensor chips such as a DNA chip (or a DNA microarray), a protein chip and the like have been recently utilized for mutational analysis of genes, analysis of SNPs (single nucleotide polymorphisms), analysis of gene expression frequency, analysis of interaction between substances, and the like. The term “sensor chip” means one wherein a diversity and multitude of substances to be detected or sensed (DNA, proteins and the like) are built up and fixed. For example, a sample is dropped over and injected into a sensor chip, thereby enabling one to exhaustively analyze if a target substance interacting with the substance for detecting is contained in the sample.

However, existing analyses using a sensor chip may not be, in most cases, sufficient with respect to accuracy and reproducibility.

To overcome the deficiency, Japanese Patent Laid-open No. 2003-279576 has proposed a method of controlling the quality of a DNA chip wherein a substance (DNA) for detection containing a fluorescent material is fixed, and an amount of the fixed substance in the DNA chip is detectable. According to this method, the quality (i.e. the fixed amount of the substance for detection) arrives at a given level, so that a DNA chip which is considered to have certain levels of detection accuracy and reproducibility can be selected.

In fact, methods of improving the detection accuracy and reproducibility have been reduced into practice, in which a plurality of sensor chips are used to analyze the same sample and the results obtained are compared with one another to evaluate the quality and performance of the sensor chips.

In the method of the above-indicated Patent Document, a problem has been left in that it is not determined whether to be fixed interactably in practice. For instance, with a DNA chip, a substance for detection is frequently fixed after synthesis and amplification of the substance through cDNA synthesizing and amplifying steps. In this case, a problem arose in that fluorescent detection is enabled in case where a nucleic acid that differs from an intended one is synthesized and amplified because the steps of cDNA synthesis and amplification are not properly performed.

With the method of evaluating the quality and performance of chips by analyzing a sample by use of a plurality of sensor chips and comparing the results with one another, a problem was left in that the case where a problem resides in the quality and performance of chip cannot be discriminated from the case where a problem is directed to the state or condition of a sample.

### SUMMARY OF THE INVENTION

Accordingly, it is an object of the invention to provide a method for judging whether to be actually fixed in an interactable manner, and also a method for evaluating if a sample is properly sampled and prepared.

It is another object of the invention to provide a sensor chip applicable to the above-mentioned methods.

We have newly discovered, as a result of investigation on alterations of circadian expressions of circadian rhythm control genes in circadian rhythm peripheral tissues (heart, lung, liver, stomach, spleen and testis) that nine circadian rhythm control genes (Bal1, Npas2, Rev-erbα, Dbp, Rev-erbβ, Per3, Per1, Per2, Cry1) have circadian expression rhythms. The expressed amounts of the nine circadian rhythm control genes in circadian rhythm peripheral tissues are measured in time series to obtain data concerning the expression levels and expression timings of the circadian rhythm control genes.

In accordance with the invention, there is provided a method for inspecting the quality of a sensor chip using either of (1) and (2) indicated below as a judgment index:

(1) interrelations of a plurality of circadian rhythm control genes in respect of expression level thereof, and

(2) time-lapse patterns of specified circadian rhythm control genes in respect of expression level thereof.

The expression level used herein means an expression level of mRNA that is a transcription product of circadian rhythm control gene in the case of a DNA chip, and an expression level of a protein that is a transcription/translation product of circadian rhythm control gene with the case of a protein chip.

For instance, a sample for quality inspection that has been prepared beforehand is analyzed with a sensor chip to obtain interrelations of a plurality of circadian rhythm control genes in respect of the expression level thereof or time-lapse patterns of specified circadian rhythm control genes in respect of the expression level thereof, followed by comparison with the data mentioned hereinabove to inspect the quality of the sensor chip, i.e. whether or not a substance for detection is actually fixed to the sensor chip in an interactable manner. In the practice of the invention, analysis is carried out for a plurality of genes or for a plurality of time points, with the attendant advantage that the accuracy of the quality inspection can be improved.

Next, in the practice of the invention, a method for sample evaluation is provided wherein whether sampling and preparation of a sample to be subjected to detection of interaction between substances is properly made is evaluated using (1) and (2) indicated below as a judgment index,

(1) Interrelations of a plurality of circadian rhythm control genes in respect of expression level thereof, and

(2) Time-lapse patterns of specific circadian rhythm control genes in respect of expression level thereof.

For instance, where a target substance-containing sample is analyzed with a sensor chip, comprehensive analyses of the target substance-containing sample with the sensor chip are made, and simultaneously, the analyses of expression levels of the circadian rhythm control genes are made at a specified region of the sensor chip. Subsequently, the interrelations in respect of the expression levels of a plurality of circadian rhythm control genes or the time-lapse patterns in respect of the expression levels of specific circadian rhythm control genes are compared with the data indicated hereinbefore to enable one to evaluate if sampling and preparation of the sample is proper or not.

More particularly, if proper sampling or preparation of a target substance-containing sample is not made, if the target substance is decomposed, or if the target substance is not synthesized, the expression levels of the circadian rhythm control genes cannot be detected. Hence, when the expression levels of the circadian rhythm control genes in the target substance are compared with the afore-indicated data, it can be determined whether the target substance is decomposed. Besides, analysis is made, according to the invention, with respect to a plurality of genes or a plurality of time points as stated hereinbefore, so that the accuracy of sample evaluation can be conveniently improved.

The above-stated methods are all applicable to DNA chips or protein chips. According to the invention, there is also provided a DNA chip wherein cDNA, which has been synthesized through reverse transcription from mRNA that is a transcription product of circadian rhythm control gene, is fixed to a substrate region. This substrate region is used as a quality inspection region or a sample evaluation region of the sensor chip. Moreover, there is provided a protein chip wherein a protein that interacts with a protein, which is a transcription/translation product of circadian rhythm control gene, is fixed to a specified substrate region, and this specified substrate region is used as a quality inspection region or a sample evaluation region of the sensor chip.

It is to be noted that the base sequences of the circadian rhythm control genes according to the invention have been laid open as the public database of NCBI (National Center for Biotechnology Information). The gene numbers of the respective genes in the database of NCBI are indicated below wherein each figure in parentheses indicates a region in gene.

(1) Bmal1; human NT_{—}009237 (12054318 to 12069318) mouse NT_{—}081129.1 (107781 to 122781), rat NW_{—}047562.1 (13774073 to 13789073). (2) Npas2: human hCG27614 (95632226 to 65646226), mouse mCG8437 (35980102 to 35994102), rat rCT22431 (39204499 to 39218499). (3) Rev-erbα: human hCG93862 (34926094 to 34912094), mouse mCG15360 (105438925 to 105424925), rat rCG33292 (82492796 to 82478796). (4) Dbp: human NT_{—}011109.15 (c21417778 to 21402778), mouse NT_{—}078442.1 (59711 to 74711), rat NW_{—}047558.1 (5120734 to 5135734). (5) Per3: human NT_{—}021937.16 (1962822 to 1977822), mouse NT_{—}039268.2 (c4331528 to 4316528), rat NW_{—}047727.1 (c801656 to 8001956). (6) Per1: human NT_{—}010718.14 (c6905708 to 6890708), mouse NT_{—}039515.2 (65661216 to 65676216), rat rCG34390 (52960430 to 52974430). (7) Per2: human NT_{—}005120.14 (c5136562 to 5121562), mouse NT_{—}039173.2 (c5833757 to 5818757), rat NW_{—}047817.1 (c6827703 to 6812703).

The technical terms used herein are defined below.

The term “circadian rhythm control gene cluster” generically means genes related to a control mechanism of circadian rhythm in vivo. The term “circadian rhythm control gene” means genes related to a control mechanism of circadian rhythm.

The term “sensor chip” means a DNA chip, a protein chip and the like chips wherein diversity and multitude of detection substances are built up and fixed. The term “detection substance” means a substance, such as a nucleic acid, a protein or the like, which interacts with a target substance. The term “target substance” means a substance specifically interacting with the detection substance.

The term “interrelation” means a certain relation between two or plural amounts or levels.

The term “circadian rhythm peripheral tissue” means tissues other than a circadian rhythm center in vivo (suprachiasmatic nucleus (SCN) of the hypothalamic area of brain) and include, for example, heart, lung, liver, stomach, spleen, kidney and the like.

According to the invention, the quality of a sensor chip is inspected to determine whether to be interactably fix or not in practice. Moreover, according to the invention, whether sampling and preparation of a sample is properly made can be evaluated.

### BRIEF DESCRIPTION OF THE DRAWINGS

FIG. 1 is a view showing the expression level of each of circadian rhythm control genes for different circadian times and organs;

FIG. 2 is a perspective view showing an outer appearance of an instance of a sensor chip;

FIG. 3 is a view showing an expression level of each circadian rhythm control gene for different circadian times and organs;

FIG. 4 is a view showing an expression level of Per1 gene for different circadian times and organs;

FIG. 5 is a view showing an expression level of Per2 gene for different circadian times and organs;

FIG. 6 is a view showing an expression level of Per3 gene for different circadian times and organs;

FIG. 7 is a view showing an expression level of Bmal1 gene for different circadian times and organs;

FIG. 8 is a view showing an expression level of Npas2 gene for different circadian times and organs;

FIG. 9 is a view showing an expression level of Dbp gene for different circadian times and organs;

FIG. 10 is a view showing an expression level of Rev-erbα gene for different circadian times and organs;

FIG. 11 is a view showing an expression level of Rev-erbβ gene for different circadian times and organs;

FIG. 12 is a view showing an expression level of Cry1 gene for different circadian times and organs; and

FIG. 13 is a graph showing the results of Example 2. Sequence Table: 200407051916228750_A163_{—}0490406704_{—}12004198619_AAA_{—}0.app

### DETAILED DESCRIPTION OF THE PREFERRED EMBODIMENTS

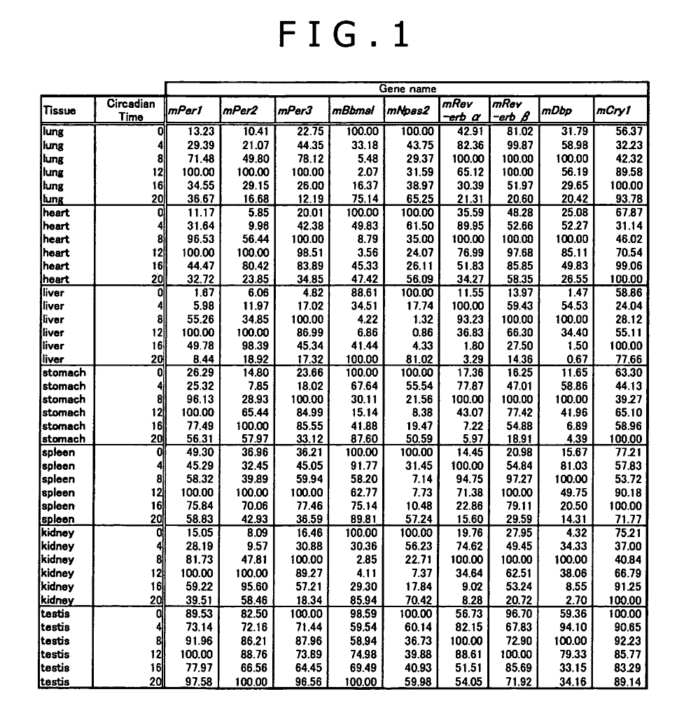

We have investigated changes in circadian expression of circadian rhythm control genes in circadian rhythm peripheral organs (heart, lung, liver, stomach, spleen and testis) and, as a result, newly found that nine circadian rhythm control genes (Bal1, Npas2, Rev-erb α, Dbp, Rev-erbβ, Per3, Per1, Per2, Cry1) have circadian expression rhythms, respectively. As shown in FIG. 1, the expression level and expression timing of each of the nine circadian rhythm control genes were measured in time series to obtain data related to the expression levels and timings of the circadian rhythm control genes (see Example 1 appearing hereinafter with respect to test procedures).

This data serves as standard bases of circadian expression rhythms of circadian rhythm control genes in peripheral tissues. Accordingly, when comparing the expression levels of the respective circadian rhythm control genes, the interrelations among the expression levels of plural circadian rhythm control genes and a time-lapse pattern in an expression level of a specified circadian rhythm control gene with those values that are obtained through analysis after dropping over or injecting into a sample in a sensor chip, the quality inspection of the sensor chip and the evaluation of a collected and prepared sample can be made, for example.

For a quality inspection method of a sensor chip embodying the invention, an instance of a quality inspection method of a DNA chip is now illustrated below.

Initially, at a manufacturing stage of a sensor chip, a diversity and a great number of cDNA's are fixed to a substrate region of a sensor chip for the purpose of comprehensive analysis of interrelations thereof. At the same time, cDNA's obtained by synthesis through reverse transcription from mRNA that is a transcription product of individual circadian rhythm control gene are, for example, fixed to a specified substrate region of the sensor chip.

In this embodiment, cDNA's wherein nine circadian rhythm control genes (Bal1, Npas2, Rev-erbα, Dbp, Rev-erb β, Per3, Per1, Per2, Cry1) are coded are, respectively, fixed to a specified substrate region of the sensor chip. Although all of the nine circadian rhythm control genes need not be fixed, it is preferred that a plurality of circadian rhythm control genes selected from the nine genes are fixed. In order to check a time-lapse pattern of expression level, it is preferred to provide a plurality of regions where the same cDNA is fixed.

On the other hand, a sample for quality inspection is provided. The quality inspection sample is preferably cDNA that is synthesized by sampling any of organs such as heart, lung, liver, stomach, spleen and testis, extracting mRNA from the cells of the sampled organ and subjecting to reverse transcription from the mRNA. The sampling time of the organ should preferably be any of 0 o'clock, 4, o'clock, 8 o'clock, 12 o'clock, 16 o'clock, 20 o'clock and 24 o'clock in terms of circadian time (CT). The term “circadian time” is intended to mean a time provided that a light conditions-commencing time is defined as 0 o'clock (herein and whenever it appears hereinafter).

Next, the sample for quality inspection is dropped over or injected into the specified substrate region where cDNA's of the circadian rhythm control genes are fixed. Subsequently, the hybridization between the cDNA's of the circadian rhythm control genes and the cDNA's in the sample is sensed to measure expression levels of the circadian rhythm control genes. The expression levels of the circadian rhythm control genes in the sample are compared with the afore-indicated data to carry out the quality inspection of the sensor chip.

For instance, as a result of quality inspection using a sample for quality inspection prepared from the heart sampled at the circadian time of 8 o'clock, if expressions of Per1 and Per2 are detected, it can be determined that a substance for detection is fixed to the sensor chip interactably with a target substance because of the detection of the expression of Per1 or Per2.

Moreover, where the expression levels of Pr1 and Per2 indicate the same interrelation with the afore-indicated data, an amount of fixation to the sensor chip can be presumed. In the data, the expression level of Per1 in the heart sampled at the circadian time of 8 o'clock is at 96.53%, and the expression level of Per2 is at 56.44%. If the expression level of Per1 and the expression level of Per2 are in interrelation with each other even though the results of the quality inspection are lower than the values of the data, it can be presumed that although an amount of a fixed substance for detection is merely small, fixation to the sensor chip is made normally. In addition, the amount of the fixed substance for detection can be estimated through comparison with the data.

Similarly, as a result, for example, of quality inspection using a sample for quality inspection prepared form the heart sampled at the circadian time of 8 o'clock and a sample for quality inspection prepared from the heart sampled at the circadian time of 12 o'clock, if Per1 is sensed from both samples, it can be determined that a substance for detection is fixed to the sensor chip interactably with a target substance owing to the fact that the expression of Per1 has been detected.

Further, an amount of a fixed substance for detection can be estimated through the comparison between the expression levels of Per1 of those sampled at the circadian time of 8 o'clock and at the circadian time of 12 o'clock (or through the time-lapse analysis of patterns of the expression levels of Per1).

It will be noted that with a protein chip, the quality inspection can be made in a way similar to that set out hereinabove. In this case, a target protein for detection of interaction is fixed to a sensor chip, and at the same time, for example, a protein (such as an antibody) interactable with a protein that is a transcription/translation product of a circadian rhythm control gene is fixed to a specified substrate region. The expression level of the protein, which is the transcription/translation product of the circadian rhythm control gene, in the sensor chip for quality inspection is sensed with the sensor chip in a like manner as set out hereinabove. Thereafter, the interrelation and time-lapse pattern of the expression level of the circadian rhythm control gene is compared with FIG. 1, thereby enabling the inspection of the sensor chip quality.

A sample evaluating method embodying the invention is illustrated with respect to a sample evaluating method using a DNA chip.

In a manner as described hereinbefore, a diversity and a great number of cDNA's for the purpose of comprehensive analyses of interaction are fixed to a substrate region of a sensor chip at a manufacturing stage of the sensor chip, and at the same time, for example, cDNA, which has been synthesized through reverse transcription from mRNA of a transcription product of each circadian rhythm control gene, is fixed to a specified substrate region of the sensor chip. The cDNA's synthesized from the transcription products of the circadian rhythm control genes and fixed to the sensor chip are similar to those set out hereinbefore.

Next, sampling and preparation of a target substance-containing sample is made. The target substance-containing sample is prepared, for example, by sampling any of organs such as heart, lung, liver, stomach, spleen and testis, extracting mRNA from the cells of the organ, and synthesizing cDNA through reverse transcription of the mRNA. The sampling time of the organ should preferably be any of 0 o'clock, 4 o'clock, 8 o'clock, 12 o'clock, 16 o'clock, 20 o'clock and 24 o'clock in terms of circadian time (CT).

Next, a target substance-containing sample is dropped over or injected into a sensor chip. Simultaneously with the detection of a target substance in the target substance-containing sample with the sensor chip, the hybridization between cDNA's of the circadian rhythm control genes fixed to the sensor chip and cDNA's synthesized through reverse transcription from the transcription products of the circadian rhythm control genes in the target substance-containing sample is detected. The expression levels of the circadian rhythm control genes detected with the sensor chip are compared with the afore-indicated data to evaluate if the sampling or preparation of the sample is proper.

For instance, where an organ is sampled at a given time and cDNA is prepared by synthesis through reverse transcription from mRNA expressed in the organ to provide a target substance-containing sample, the cDNA's synthesized through reverse transcription from the transcription products of the circadian rhythm control genes are contained in the target substance-containing sample. To cope with this, when the target substance-containing sample is dropped over or injected into the sensor chip, the target substance-containing sample is simultaneously dropped over and injected into a region of the sensor chip where DNA's of the circadian rhythm control genes have been fixed. Then, the expression levels of the circadian rhythm control genes in the target substance-containing sample are compared with the data, so that whether the sampling and purification of the sample, particularly, an amplification step or reverse transcription step, is carried out properly can be evaluated.

For example, in the amplification or reverse transcription step for preparing a target substance-containing sample, an amount of cDNA in the target substance-containing sample may often vary. In this case, the interrelations in the expression level of a plurality of circadian rhythm control genes in the target substance-containing sample, or the time-lapse pattern in the expression level of a specified circadian rhythm control gene is compared with the data. The comparison permits a case where an amount of synthesized cDNA is merely small and a case where preparation of a target substance-containing sample is not proper to be clearly discriminated from each other.

It will be noted that with a protein chip, sample evaluation can be made in a similar manner. More particularly, when a target substance-containing sample is comprehensively analyzed by use of a sensor chip, the expression levels of proteins expressed from circadian rhythm control genes in the target substance-containing sample are compared with the data, it can be evaluated whether the target substance-containing sample is properly sampled and purified.

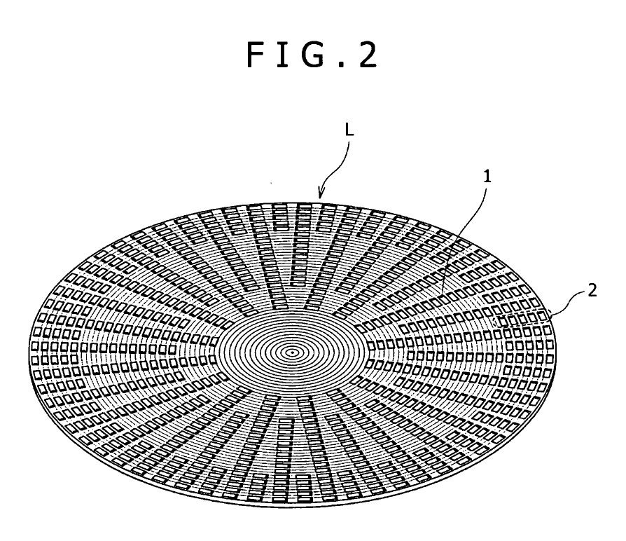

Referring now to FIG. 2, a sensor chip, such as a DNA chip, according to the invention is illustrated.

A sensor chip shown in FIG. 2 is so configured that a plurality of interaction detecting units 1 are peripherally, spirally and radially arranged on a substrate L which is in a form of a disc such as CD. The interacion detecting units 1 of the sensor chip of FIG. 2 is in a well form wherein a substance for detection, cDNA synthesized through reverse transcription from mRNA that is a transcription product of circadian rhytm control gene, and a protein interacting with a protein that is a transcription/translation product of circadian rhythm control gene, and the like can be fixed within the well.

The substrate L may be formed of such a material as used for optical information recording media such as CD (Compact Disc), DVD (Digital Versatile Disc), MD (Mini Disc) and the like. The shape of he substrate L is not limited to such a disc as shown in FIG. 2 and may be changed freely depending on the purpose. It will be noted that if the substrate L is formed of an inexpensive synthetic resin, running costs for the manufacture of sensor chip can be suppressed to be lower than those for conventionally employed glass chips.

Where the sensor chip consists of a DNA chip, cDNA synthesized through reverse transcription from mRNA that is a transcription product of circadian rhythm control gene is, for example, fixed to a specified substrate region 2 of the substrate L. This substrate region 2 may be used as a quality inspection region or sample evaluation region of the sensor chip.

Likewise, where the sensor chip consists of a protein chip, a protein interacting with a protein that is a transcription/translation product of circadian rhythm control gene is fixed to the specified substrate region 2. This substrate region 2 may be used as a quality inspection region or sample evaluation region of the chip.

The invention is more particularly described by way of examples.

### EXAMPLE 1

In Example 1, expression peaks of circadian rhythm control genes in individual circadian rhythm peripheral tissues at clock-time intervals were checked. The procedure was as follows.

Initially, a circadian rhythm of a mouse used for an experiment was synchronized. More particularly, a mouse was bred over 2 weeks in a room which was kept under light conditions from 8 to 20 o'clock and under dark conditions from 20 to 8 o'clock of the next day so that the circadian rhythm of the mouse was synchronized. It will be noted that for the experiment, ICR mouse (male, 5-week old) purchased from Nihon SLC Kabushikikaisha was used.

Next, the heart, lung, liver, stomach, spleen and testis were, respectively, sampled from the mouse at given time intervals as a circadian rhythm peripheral tissue and immediately frozen with liquid nitrogen. It will be noted that the sampling time for the respective internal organs was so set as to be every four hours from 8 o'clock determined immediately after the synchronization of the circadian rhythm (8 o'clock, 12 o'clock, 16 o'clock, 20 o'clock, 24 o'clock and 4 o'clock of the next day).

Thereafter, the sampled organs were, respectively, homogenized, followed by extraction of total RNA from individual organs by use of Promega Total SV RNA Isolation Kit (made by Promega Inc.). Next, quantitative real time RT-PCR (quantitative real time reverse transcription polymerase chain reaction) was carried out to measure expressed amounts of circadian rhythm control genes.

The thus measured circadian rhythm control genes were 14 in number as indicated below. Bal1, Npas2, Rev-erbα, Dbp, Rev-erbβ, Per3, Per1, Per2, Cry1, Cry2, Clock, Ck1δ, Ck1ε and Tim.

It will be noted that quantitative real time RT-PCR used was ABI PRISM7000 (made by Applied Biosystems Inc.). For PCR, 40 cycles of 50° C., 2 minutes/95° C., 10 minutes/(95° C., 15 seconds/60° C., 1 minute) were carried out using SYBR Green PCR Master Mix (ABI Inc.). The primers used are indicated below. Bal1 (sense primer: sequence No. 1, antisense primer: sequence No. 2), Npas2 (sense primer: sequence No. 3, antisense No. 4), Rev-erbα (sense primer: sequence No. 5, antisense No. 6), Dbp (sense primer: sequence No. 7, antisense No. 8), Rev-erbβ (sense primer: sequence No. 9, antisense No. 10), Per3 (sense primer: sequence No. 11, antisense No. 12), Per1 (sense primer: sequence No. 13, antisense No. 14), Per2 (sense primer: sequence No. 15, antisense No. 16), Cry1 (sense primer: sequence No. 17, antisense No. 18), Cry2 (sense primer: sequence No. 19, antisense No. 20), Clock (sense primer: sequence No. 21, antisense No. 22), Ck1δ (sense primer: sequence No. 23, antisense No. 24), Ck1ε (sense primer: sequence No. 25, antisense No. 26), and Tim (sense primer: sequence No. 27, antisense No. 28).

In the above experiment, the expression level of the respective genes is a relative value calculated based on the expressed amount of a housekeeping gene. First, transcription products of β-actin and G3PDH were simultaneously amplified as selected from the total RNA extracted at the time of reverse transcription PCR in the course of the experimental procedure, followed by synthesis of cDNA of β-actin and G3PDH. The β-actin and G3PDH were both a housekeeping gene, with expression levels being substantially constant. The expression levels of the respective genes were compared with the expression level of β-actin or G3PDH, from which a relative expression level was calculated. It is to be noted that for the calculation of expression level, two types of housekeeping genes (β-actin and G3PDH) were used to obtain expression levels of individual genes, and the accuracy of the expression level of individual genes was increased by correcting and adjusting errors involved in the case using β-actin and also in the case using G3PDH.

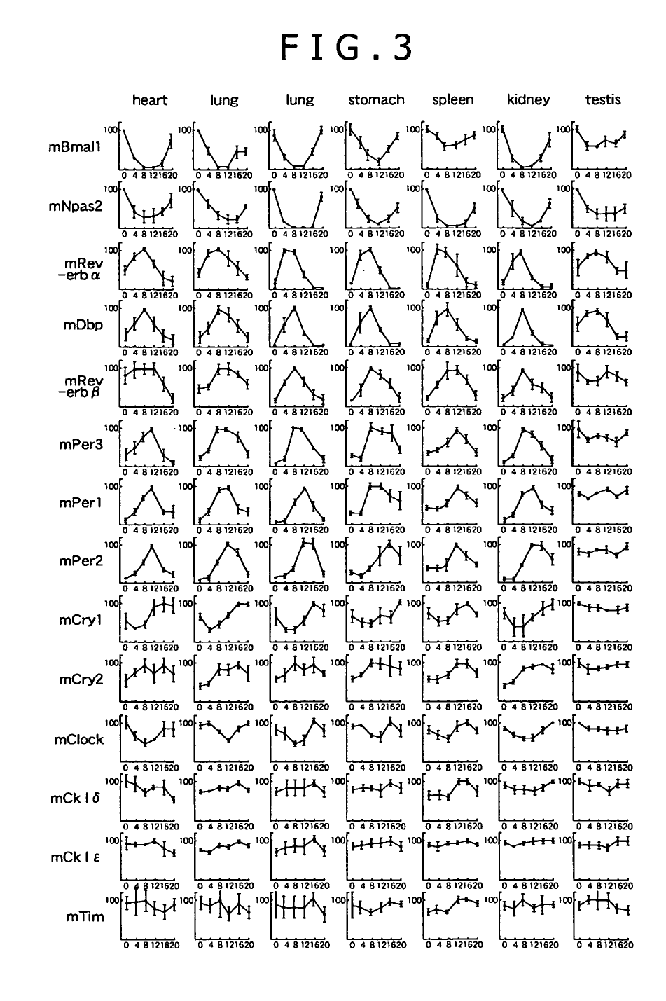

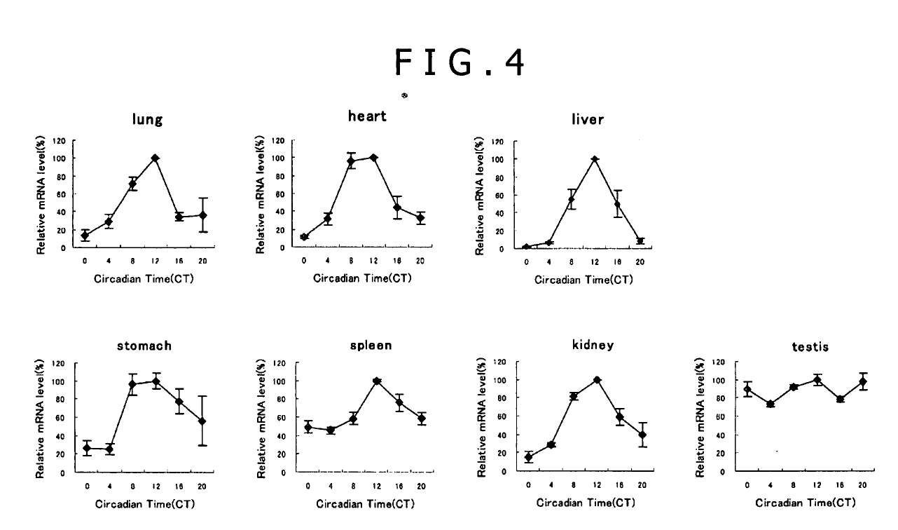

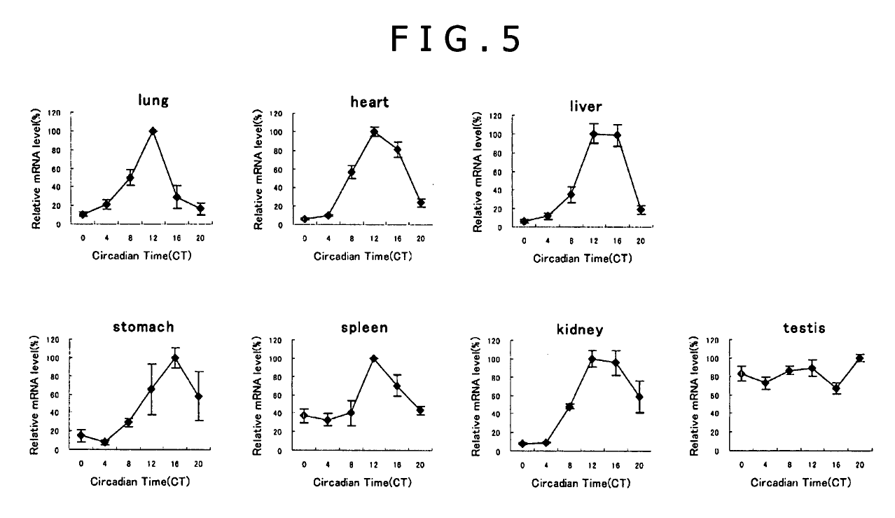

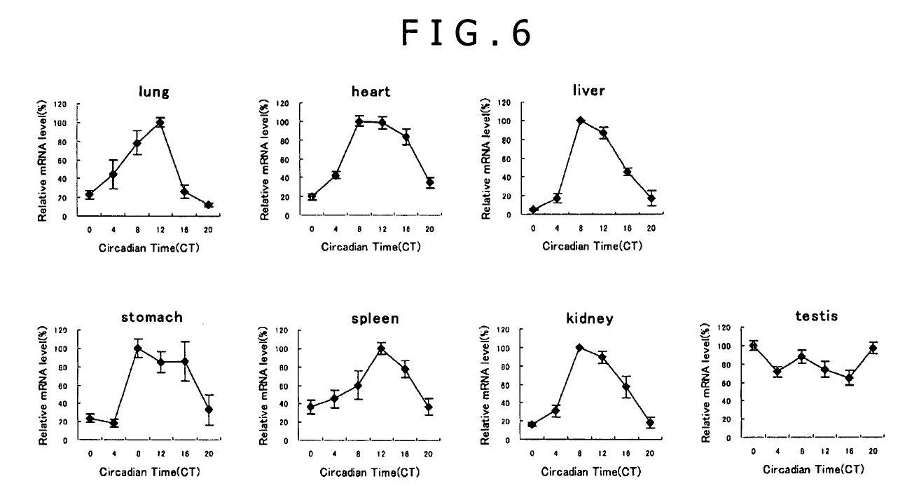

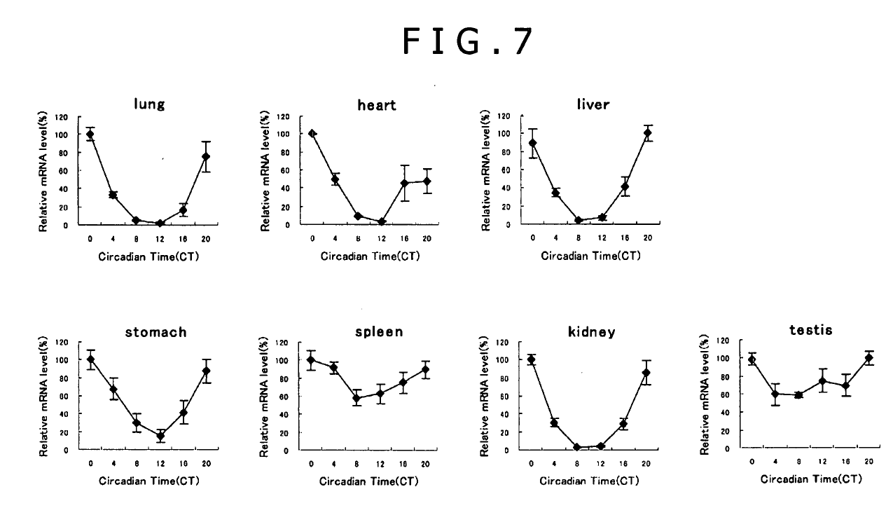

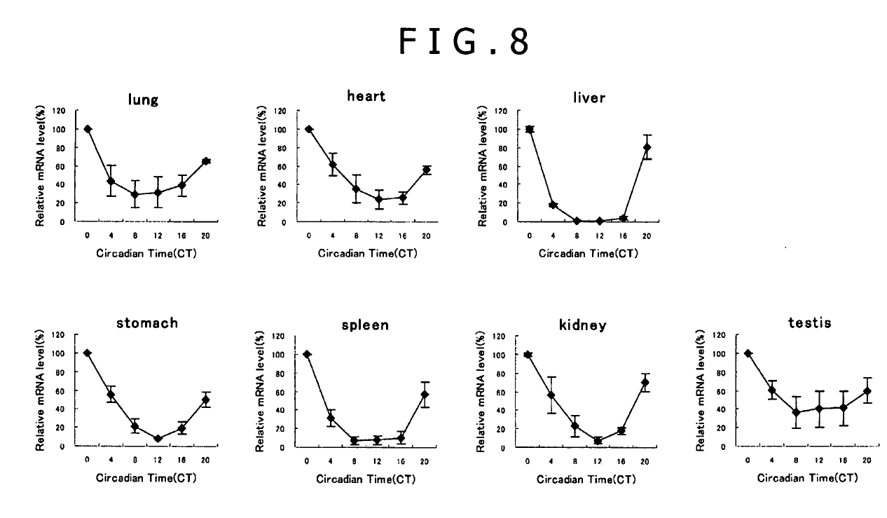

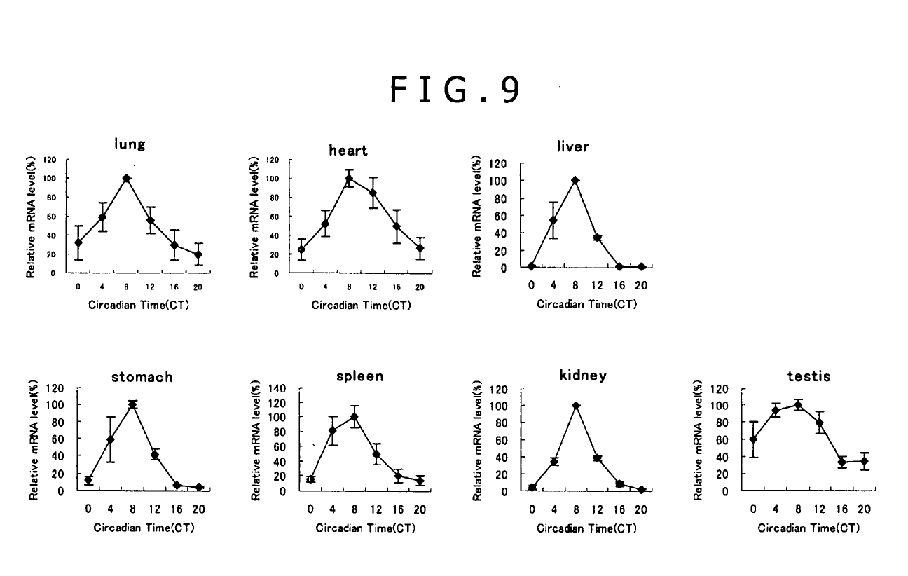

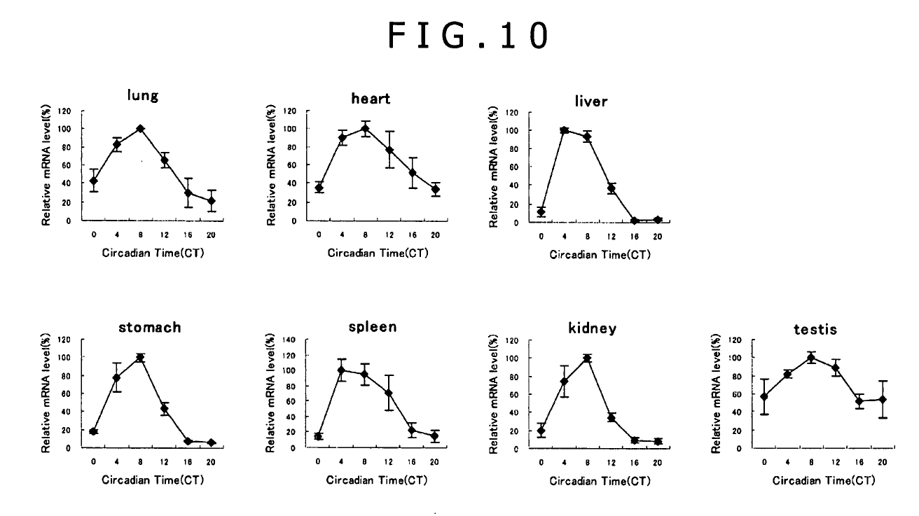

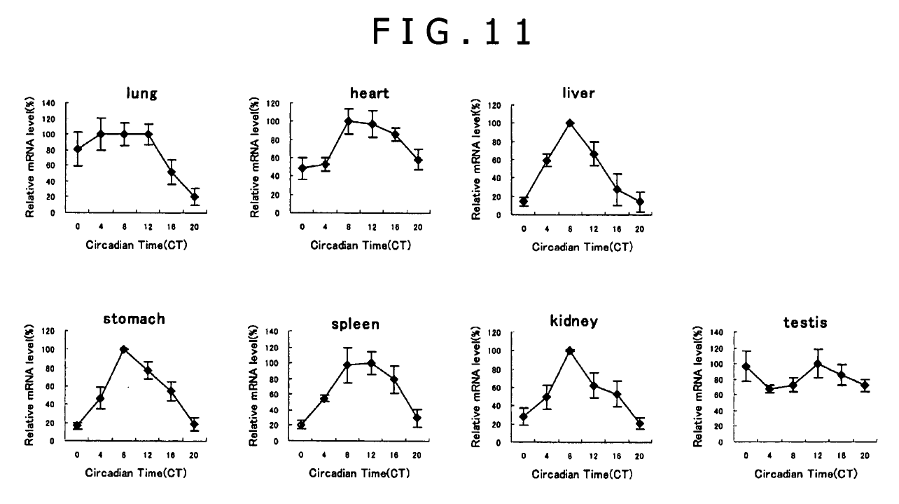

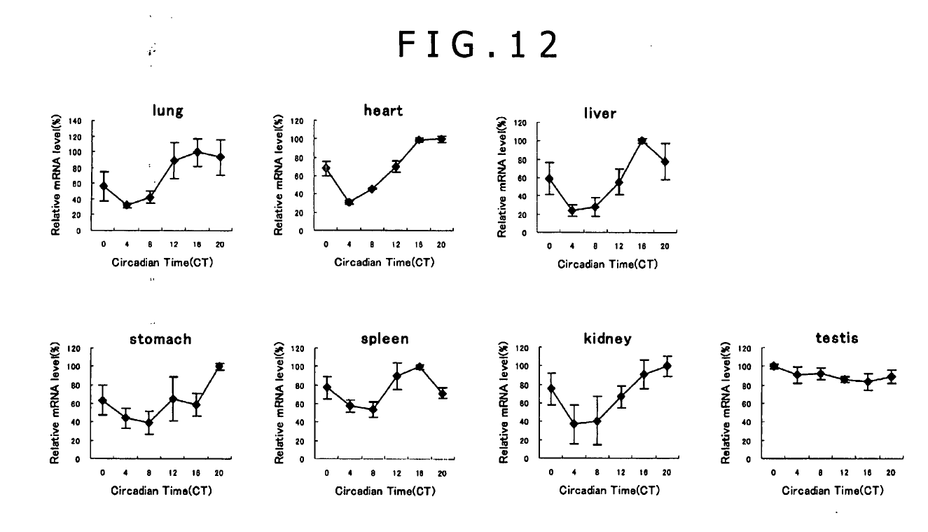

The results are shown in FIG. 3 (see FIG. 1 with respect to numerical values). In FIG. 3, there are shown the expression levels of the respective genes for heart, lung, liver, stomach, spleen, kidney and testis. The symbol “m” in the genes (e.g. initially occurring “m” in “mBall”) means mammalian. In each graph, the abscissa indicates a circadian time (CT), and the ordinate indicates a relative expression level (%). As to the circadian time (CT), CT0 to CT12 are under light conditions (subjective daytime) and CT12 to CT24 are under dark conditions (subjective night)(herein and also in FIGS. 4 to 13).

As shown in FIG. 3, the circadian expression rhythms were observed for nine genes among 14 genes (Bmal1, Npas2, Rev-erbα, Rev-erbβ, Dbp, Per3, Per1, Per2 and Cry 1) in all the organs except for testis.

It is to be noted that FIGS. 4 to 12 are, respectively, graphs showing the results of FIG. 3 for different types of circadian rhythm control genes. That is, FIGS. 4 to 12, respectively, show the results for Per1, Per2, Per3, Bal1, Npas2, Dbp, Rev-erbα, Rev-erbβ and Cry 1.

### EXAMPLE 2

In Example 2, the expression level of Per1 in a sample collected and prepared from lung was compared with the afore-indicated data (see FIG. 1) to make quality inspection and sample evaluation.

Initially, like Example 1, the circadian rhythms of a mouse used for this experiment were synchronized. Thereafter, the lung was sampled from each mouse at given times, followed by immediate freezing with liquid nitrogen. The sampling time o'clock was set at every four hours (CT0, CT4, CT8, CT12, CT16, CT20) immediately after (CT0) the synchronization of the circadian rhythms. It will be noted that the symbol CT means a circadian time wherein a light condition-commencing time is set at 0 o'clock (CT0).

Next, the respective ling samples were homogenized, after which total RNA was extracted from the respective organs by use of Promega Total SV RNA Isolation Kit (Promega Inc.). Next, quantitative real time RT-PCR (quantitative real time reverse polymerase reaction) was carried out to measure expressed amounts of circadian rhythm control genes. It is to be noted that as set out hereinbefore, the expression levels of individual genes in this experiment are relative values calculated based on the expressed amount of housekeeping genes.

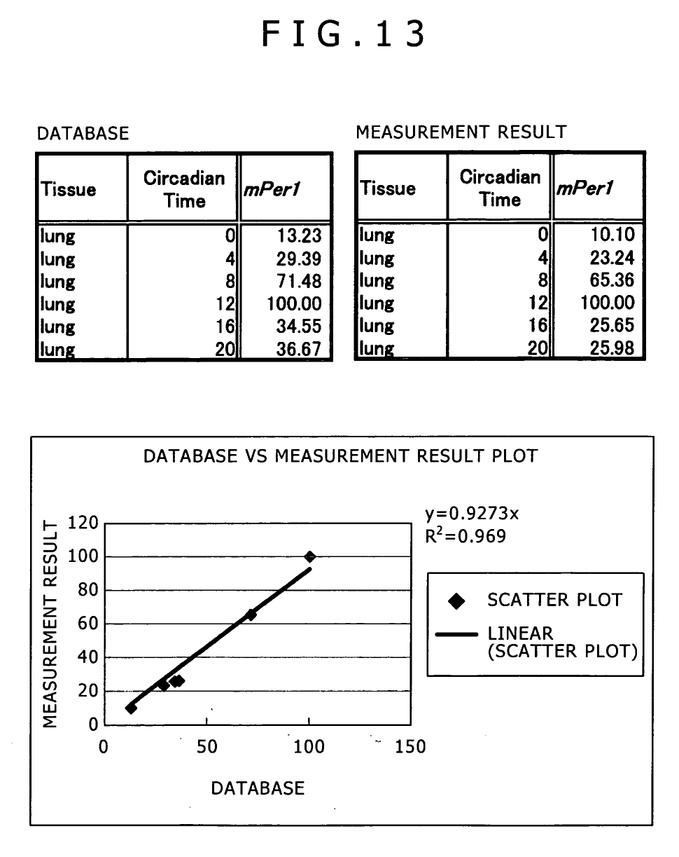

The results are shown in FIG. 13. In FIG. 13, the term “database” appearing at the upper left means values of the afore-indicated data. The term “results of measurement” appearing at the upper right means the results of measurement according to this experiment. In FIG. 13, the lower graph shows the interrelation between the values of “database” and the value of “results of measurement”. The abscissa of the graph indicates the values of “database” and the ordinate indicates the values of “results of measurement”.

As shown in FIG. 13, the values of “database” and the values of “results of measurement” are not coincident but interrelate with each other. More particularly, the results of this experiment reveal that not only the comparison of time-lapse patterns of expression levels of Per1 enables one to determine that a substance for detection is fixed to a sensor chip intractably with a target substance, but also the fixed amount of a substance for detection can be predicted. The results of the experiment show that it can be determined whether sampling and purification of samples, particularly, amplification and reverse transcription steps, are carried out properly even when an amount of DNA varies in a target substance-containing sample.

When an inspection method of a sensor chip according to the invention is used, the quality and performance of the sensor chip can be maintained at a certain level. The sample evaluation method of the invention enables one to determine whether preparation of samples is proper, simultaneously with analyses using an intended sensor chip. Thus, the invention is industrially useful.

What is claimed is:

1. A method for inspecting a quality of a sensor chip using either of (1) and (2) indicated below as a judgment index:
(1) interrelations of a plurality of circadian rhythm control genes in respect of expression level thereof, and
(2) time-lapse patterns of specified circadian rhythm control genes in respect of expression level thereof.

2. The method for inspecting a quality of a sensor chip according to claim 1, wherein said expression level consists of an expression level of mRNA that is made of a transcription product of a circadian rhythm control gene, and said sensor chip is made of a DNA chip.

3. The method for inspecting a quality of a sensor chip according to claim 1, wherein said expression level consists of an expression level of a protein that is made of a transcription/translation product of a circadian rhythm control gene, and said sensor chip is made of a protein chip.

4. A method for sample evaluation wherein whether sampling and preparation of a sample to be subjected to detection of interaction between substances is properly made is evaluated using the following factors (1) and (2) as a judgment index,
(1) interrelations of a plurality of circadian rhythm control genes in respect of expression level thereof, and
(2) time-lapse patterns of specific circadian rhythm control genes in respect of expression level thereof.

5. The method for sample evaluation according to claim 4, wherein said sample is made of a target substance-containing sample dropped over or injected into a region to which a detecting substance of a sensor chip is fixed.

6. The method for sample evaluation according to claim 5, wherein absence or presence of decomposition of said target substance is determined.

7. The method for sample evaluation according to claim 5, wherein said sensor chip is made of a DNA chip or protein chip.

8. A DNA chip comprising a substrate having a specified substrate region where cDNA synthesized through reverse transcription from mRNA that is a transcription product of a circadian rhythm control gene is fixed, said substrate region being used as a quality inspection region or sample evaluation region of said chip.

9. A protein chip comprising a substrate having a specified substrate region where a protein intereactable with a protein that is a transcription/translation product of a circadian rhythm control gene is fixed, said substrate region being used as a quality inspection region or sample evaluation region of said chip.
